(19) United States
(12) Patent Application Publication
(10) Pub. No.: US 2015/0148875 A1
(43) Pub. Date: May 28, 2015

(54) Toe Tunic For Big Toe Pain

(71) Applicant: Judith Kathleen Knez, Bokeelia, FL (US)

(72) Inventor: Judith Kathleen Knez, Bokeelia, FL (US)

(21) Appl. No.: 14/090,357

(22) Filed: Nov. 26, 2013

(51) Int. Cl. A61F 7/02

(52) U.S. Cl. CPC A61F 7/02; A61F 2007/0046
USPC 607/111

(57) ABSTRACT

The Toe Tunic is a device with a non-toxic filling that is specifically designed to help reduce the pain and swelling associated with the big toe, and big toe joint injuries, diseases, disorders and ailments. It is a pull-on apparatus that covers the big toe and big toe joint leaving other toes exposed, easily pulls onto the foot, and delivers hot or cold therapy to the big toe area as medically indicated. It will be helpful in reducing pain of common sports injuries to the big toe (Turf Toe) as well as for other big toe injuries, arthritis, sprains, strains and numerous other big toe-related disorders. While there are ankle and foot products available on the market, this invention is unique in that it targets pain relief specifically for the big toe and big toe joint.

### CROSS-REFERENCE TO RELATED APPLICATION

Reference Provisional Patent Application No. 61/806,106 filed on Mar. 28, 2013 by applicant, Judith Kathleen Knez entitled Toe Tunic.

### BACKGROUND OF THE INVENTION

Swelling and pain associated with strains, sprains, torn ligaments, arthritis, injury and other maladies can specifically affect the big toe and the big toe or Metatarsophalangeal (MTP) joint. Many field sports players sustain injury to this particular area of the foot. Treatment for big toe and MTP joint problems routinely may include rest, elevation, ice and compression. While there are existent braces, insoles, boots and other devices that focus on treatment of big toe and MTP joint problems, and there are other hot/cold devices for the ankle and foot, there is not a product that delivers hot and cold therapy specifically targeting the big toe and big toe joint, for relief of pain and swelling. It is for this reason that I invented the Toe Tunic.

### BRIEF SUMMARY OF THE INVENTION

The intent of the Toe Tunic is to help reduce pain and swelling by applying hot or cold treatment specifically to the big toe and the MTP joint just above the big toe where the majority of big toe pain emanates. The Toe Tunic design specifically targets the big toe and MTP joint areas and comfortably slips onto the foot to help reduce the pain and swelling associated with big toe injuries and disorders. The device is reusable, and filled with a non-toxic filling, that may be either heated in the microwave oven or put into a freezer to obtain the precise type of therapy medically indicated for the problem.

### BRIEF DESCRIPTION OF THE VIEW OF THE DRAWING

The drawing depicts an overhead view of the Toe Tunic as it would be seen on the foot and identifies the approximate location of the Metatarsophalangeal (MTP) joint.

### DETAILED DESCRIPTION OF THE INVENTION

The Toe Tunic is a one-piece device filled with a non-toxic filling, is fabric covered, reusable, slips onto the foot, delivers either hot or cold therapy to the big toe and the MTP joint just above the big toe, which helps provide relief for the pain and swelling associated with many big toe injuries and ailments such as, but not limited to, sprains, strains, torn ligaments, arthritis, turf toe and other big toe-related maladies.

The Toe Tunic pulls onto the foot similar to a sock, but the only toe that is encased within the tunic is the big toe, with the other four toes uncovered and exposed. The tunic band that is part of the encased big toe covering extends upwards onto the foot, covering the MTP joint where much of the pain associated with the big toe is localized, and encircles the foot just above the exposed four toes.

The Toe Tunic contains a filling that is non-toxic, and has properties that allow for both heating and freezing (dual therapy) in order to provide for the type of therapy that is recommended or medically indicated for a specific big toe problem. The tunic filling is located between the inner lining and outer covering of the Toe Tunic.

The Toe Tunic would be manufactured in compliance with good manufacturing practices as indicated in the standards and regulations of the United States Food and Drug Administration (FDA) governing medical devices intended for human use.

1. A therapeutic device for the big toe and the Metatarsophalangeal (MTP) joint to aid in the relief of pain and swelling characterized by:
A one-piece, reusable, fabric-covered, slip-on device with a non-toxic filling, that encases only the big toe and covers the Metatarsophalangeal joint [hereinafter referred to as “MTP joint”] and delivers hot or cold therapy for big toe and MTP joint injuries, diseases, ailments and disorders.

2. The non-toxic filling noted in claim 1, is contained between the inner lining and outer covering of the device, may be a gel, a food grade filling of other type filling with properties that allow for heating in a microwave oven or putting in a freezer prior to putting onto the foot.

3. The fabric cited in claim 1 allows for ease in slipping the device onto and off of the big toe and foot while also ensuring against any leakage of the gel substance noted in claim 2.

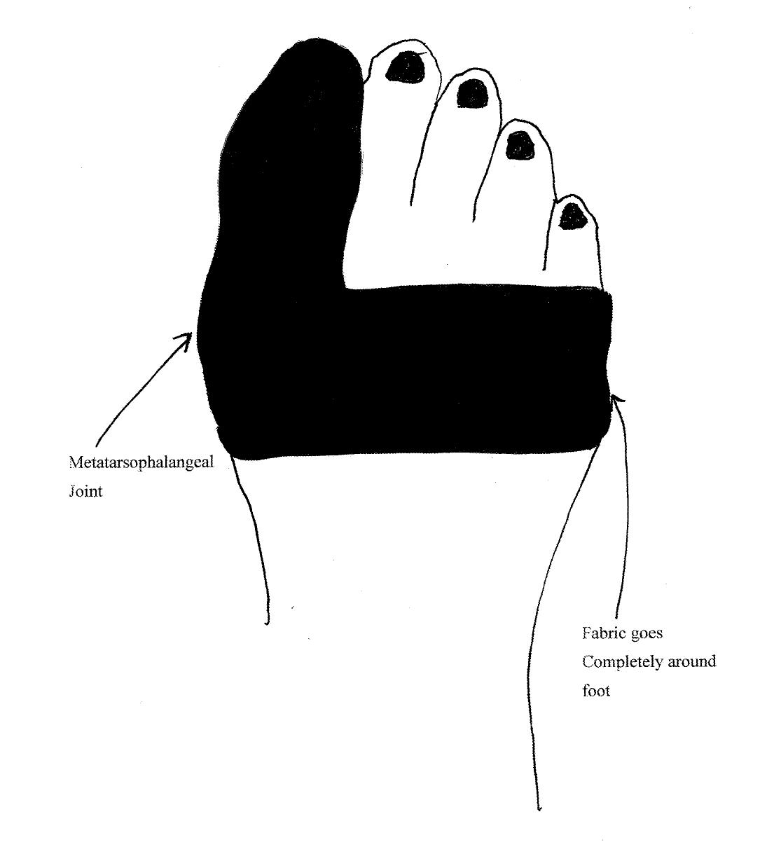

4. The device noted in claim 1 is designed as one piece to easily pull onto and encase the big toe and no other toes, while extending upward on the foot from the big toe to cover the MTP joint, and encircling the foot to hold the device in place. (FIG. 1)

5. The device noted in claim 1 is reusable.
